(19) United States
(12) Patent Application Publication
(10) Pub. No.: US 2022/0096508 A1
(43) Pub. Date: Mar. 31, 2022

(54) METHOD OF TREATMENT AND PHARMACEUTICAL DOSAGE FORM

(71) Applicant: Aprea Therapeutics AB, Solna (SE)

(72) Inventor: Karin Jorga, Solna (SE)

(21) Appl. No.: 17/428,883

(22) PCT Filed: Dec. 2, 2019
(86) PCT No.: PCT/EP2019/083302

§ 371 Date: Aug. 5, 2021

Related U.S. Application Data
(60) Provisional application No. 62/803,051, filed on Feb. 8, 2019.

(51) Int. Cl. A61K 31/706; A61K 9/00; A61P 35/00

(52) U.S. Cl. CPC A61K 31/706; A61P 35/00; A61K 9/0019

(57) ABSTRACT

The invention relates to a method of treatment of neoplastic disease in a patient with mutant TP53 with a combination of APR-246 and azacitidine, in which APR-246 is given as a fixed dose. The invention also relates to pharmaceutical dosage forms of APR-246 which employ such fixed dose.

### FIELD OF THE INVENTION

The invention relates to a method of treatment of neoplastic disease in a patient with mutant TP53 with a combination of APR-246 and azacitidine, in which APR-246 is given as a fixed dose. The invention also relates to pharmaceutical dosage forms of APR-246 which employ such fixed dose.

### BACKGROUND

APR-246 is a novel small molecule anti-cancer compound that reactivates non-functional p53 and targets the cellular redox balance, resulting in induction of apoptosis in tumor cells (Bykov et al (2016), Front Oncol 6:21). APR-246, sometimes denoted PRIMA-1Met in the literature, is a chemically synthesized prodrug, which spontaneously decays into the active moiety MQ (2-methylene-quinuclidin-3-one). APR-246, 2-hydroxymethyl-2-methoxymethyl-1-azabicyclo [2,2,2] octan-3-one, is a candidate drug in clinical development by Aprea Therapeutics AB, and has the structural formula (I):

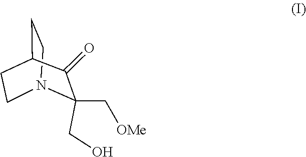 [SMILES: COCC1(CO)C(=O)C2CCN1CC2]

Based on its combined mechanism of action via p53 reactivation and the redox system, APR-246 treatment may conceivably be effective in a broad range of clinical contexts and preclinical studies have identified potential for synergy with several different anti-cancer drugs (Bykov et al (2016), supra). The ongoing clinical development program includes combination treatments where the APR-246 dosing schedule is aligned with the dosing schedule of the concurrent anti-cancer therapy. An overview of clinical studies with APR-246 is provided in the table below.

| | | Reference/ |
|---|---|---|
| | | clinicaltrials.gov |
| Study | Status | identifier |
| Monotherapy: | | |
| APR-246-01 - Targeting p53 in Vivo: A First-in- | Completed | Lehmann 2012¹ |
| Human Study With p53-Targeting Compound APR- | | |
| 246 in Refractory Hematologic Malignancies and | | |
| Prostate Cancer | | |
| APR-246-01, Amendment No. 6 - An open-label | Completed | Deneberg 2016² |
| phase I dose-finding study of APR-246 in | | |
| hematological malignancies | | |
| Combination treatments: | | |
| APR-407 (PiSARRO) - p53 Suppressor Activation | Ongoing | NCT02098343 |
| in Recurrent High Grade Serous Ovarian Cancer, a | | |
| Phase Ib/II Study of Systemic Carboplatin/ | | |
| Pegylated Liposomal Doxorubicin Combination | | |
| Chemotherapy With or Without APR-246 | | |
| APR-486 (PiSARRO-R) - p53 Suppressor | Ongoing | NCT03268382 |
| Activation in Platinum-Resistant High Grade | | |
| Serous Ovarian Cancer, a Phase 2 Study of | | |
| Systemic Pegylated Liposomal Doxorubicin | | |
| Chemotherapy With APR-246 | | |
| 16/012 (APROC) - A phase 1b/2 study evaluating | Ongoing | NCT02999893 |
| the efficacy of APR-246, a first-in-class agent | | |
| targeting mutant p53 in the treatment of platinum | | |
| resistant advanced and metastatic oesophageal or | | |
| gastro-oesophageal junction cancers | | |
| MCC-18973 - A Phase 1b/2 Study to Evaluate the | Ongoing | NCT03072043 |
| Safety and Efficacy of APR-246 in Combination | | |
| with Azacitidine for the Treatment of TP53 Mutant | | |
| Myeloid Neoplasm | | |
| GFM-APR246 - A Phase 1b/2 Study to Evaluate | Ongoing | NCT03588078 |
| the Safety and Efficacy of APR-246 in Combination | | |
| With Azacitidine for the Treatment of TP53 Mutant | | |
| Myeloid Neoplasms | | |
| A18-15331 - A Phase 3 Multicenter, Randomized, | Initiated in | NCT03745716 |
| Open Label Study of APR-246 in Combination with | the US | |
| Azacitidine Versus Azacitidine Alone for the | December | |
| Treatment of TP53 Mutant Myelodysplastic | 2018 | |
| Syndromes | | |

¹Lehmann et al (2012), J Clin Oncol 30(29): 3633-3639
²Deneberg et al(2016), Blood Cancer J 6(7): e447

Despite the advances made in clinical trials concerning the use of APR-246 alone and in combination with other active ingredients, there is a continued need to provide novel treatment options using APR-246 against malignant diseases involving TP53.

### SUMMARY OF THE INVENTION

To meet this need and/or other objectives that are evident to a skilled person from reading the present disclosure, the invention provides, in a first aspect, a method of treatment of a neoplastic disease in a patient carrying a mutant TP53 gene, comprising
administration to said patient of APR-246 at a fixed dose within the interval 2.7-7.5 g, and
administration to said patient of azacitidine at a therapeutically effective dose.

Thus, it was unexpectedly found by the inventors, in the course of clinical trials using APR-246 in combination with azacitidine, that the pharmacokinetics of APR-246 enable the use of a fixed dose of the compound within the interval 2.7-7.5 g, rather than a dose which is related to either the “lean body mass” (LBM) or the total body weight (WT) of the patient as in previous trials with the same active compound. As such, a fixed dose offers numerous benefits, not least reducing the risk of dosing errors and ease of preparation and administration. In one embodiment, the fixed dose is within the interval 3.5-6.0 g, such as within the interval 4.0-5.0 g, for example a fixed dose of approximately 4.5 g.

In the disclosed method, APR-246 administration is combined with administration of azacitidine. Azacitidine is a nucleoside analogue used mainly in the treatment of myelodysplastic syndrome, for example as approved by the US FDA since 2004. In one embodiment of the disclosed method, the therapeutically effective dose of azacitidine is a body surface area based dose within the interval 70-80 mg/m², such as 75 mg/m². In another embodiment, the therapeutically effective dose of azacitidine is a fixed dose within the interval of 100-1000 mg, such as within the interval 120-600 mg, for example a fixed dose of approximately 300 mg, or a fixed dose of approximately 200 mg.

In one embodiment of this aspect, the disclosed treatment method is carried out pursuant to the following dosing scheme over a 28-day cycle:

a) daily administration of APR-246 for four days, for example four consecutive days;

and

b) daily administration of azacitidine for at least 7 days during the 28-day cycle.

In one embodiment of this 28-day cycle dosing scheme, the administration of azacitidine is carried out during 7 consecutive days during the 28-day cycle. In a more specific embodiment, the 7 consecutive days of administration of azacitidine are selected such that there is an overlap of 1, 2, 3 or 4 days between the periods of administration of APR-246 and azacitidine. In one particular embodiment, the administration of APR-246 is carried out on days 1-4, and the administration of azacitidine is either carried out on days 1-7, or on days 2-8, or on days 3-9, or on days 4-10, during the 28-day cycle. In a specific embodiment, the administration of APR-246 is carried out on days 1-4, and the administration of azacitidine is carried out on days 4-10, during the 28-day cycle.

In an alternative embodiment, for example in the case of outpatient treatment of patients for which weekend treatment is not feasible, the administration of APR-246 is carried out on days 1-4, whereas the administration of azacitidine is carried out on 7 consecutive working days. If, in this embodiment, for example day 1 of the 28-day cycle is a Monday, APR-246 is administered on days 1-4, while azacitidine is administered on days 1-5 and days 8-9, or on days 2-5 and days 8-10, or on days 3-5 and days 8-11, or on days 4-5 and 8-12.

In another embodiment of the 28-day cycle dosing scheme, the administration of azacitidine is carried out during 14 days, for example 14 consecutive days, during the 28-day cycle. In a more specific embodiment, the 14 consecutive days of administration of azacitidine are selected such that there is an overlap of 1, 2, 3 or 4 days between the days of administration of APR-246 and the days of administration of azacitidine.

In another embodiment of the 28-day cycle dosing scheme, the administration of azacitidine is carried out during 21 days, for example 21 consecutive days, during the 28-day cycle. In a more specific embodiment, the 21 consecutive days of administration of azacitidine are selected such that there is an overlap of 1, 2, 3 or 4 days between the days of administration of APR-246 and the days of administration of azacitidine.

The route of administration of APR-246 and azacitidine is suitably selected by the attending physician based on factors such as type, species, age, weight, sex, and medical condition of the subject and the renal and hepatic function of the subject, and the particular disorder or disease being treated, as well as its severity.

In one embodiment, APR-246 is administered parenterally. In a particular such embodiment, APR-246 is administered as an intravenous infusion. In one embodiment of the disclosed method, intravenous infusion of APR-246 is carried out according to a scheme by which a substantial amount of the intended dose is infused rather quickly during the initial phase of infusion, after which the remainder of the dose is given more slowly during an extended time period. It has been found that such an infusion strategy is beneficial in keeping the Cₘₐₓ experienced by the patient during administration within a safe and well-tolerated interval. As a non-limiting example, an exemplary dose of 4.5 g of APR-246 is suitably infused over a period of six hours in such a way that around a third, i.e. 1.5 g, are given during the first 45 minutes of the period, whereas the remaining 3.0 g are infused during the rest of the period, i.e. during the remaining 5 hours and 15 minutes. The skilled person may readily adapt this schedule to the specific dose and timing requirements in a specific case. If it is necessary to carry out infusion during a relatively shorter time, for example to ensure patient compliance in a pediatric population, the fixed dose is suitably selected in the lower part of the disclosed range of 2.7-7.5 g so that administration can be completed in a shorter time without Cₘₐₓ exceeding the threshold value. In such a situation, it may be suitable to administer 2.7-4.0 g APR-246 by infusion during a period of around 4 h, rather than administering 4.5 g during 6 h.

In one embodiment, azacitidine is administered parenterally, for example as a subcutaneous injection or an intravenous infusion. In another embodiment, azacitidine is administered orally. In one embodiment of oral administration of azacitidine, azacitidine is administered as the oral formulation CC-486 (Garcia-Manero et al (2011), J Clin Oncol 29(18):2521-2527).

The disease to be treated with the method of the present disclosure is a neoplastic disease. In one embodiment, such disease is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

In a more specific embodiment, said neoplastic disease is selected from malignant neoplasms of lymphoid, hematopoietic and related tissue including multiple myeloma, lymphoid leukemia or myeloid leukemia; and neoplasms of uncertain or unknown behavior including myelodysplastic syndrome.

In a yet more specific embodiment, said neoplastic disease is myelodysplastic syndrome.

The patient to be treated by the method according to the disclosure is a carrier of a mutant TP53 gene. The genetic status of a prospective patient is suitably established by DNA sequence analysis, carried out on a sample of peripheral blood or bone marrow.

In a second aspect, the invention provides a pharmaceutical dosage form, comprising APR-246 at a fixed dose within the interval 2.7-7.5 g and at least one pharmaceutically acceptable excipient. In one embodiment, the fixed dose is within the interval 3.5-6.0 g, such as within the interval 4.0-5.0 g, for example a fixed dose of approximately 4.5 g.

In one embodiment, a pharmaceutical dosage form of APR-246 according to the second aspect of the disclosure is a composition for parenteral administration, such as for intravenous infusion. In this embodiment, possible compositions include aqueous and non-aqueous sterile injection solutions which may contain anti-oxidants, buffers or other pH-adjusting components, bacteriostats and solutes which render the composition isotonic with the blood of the intended recipient; and aqueous and non-aqueous sterile suspensions which may include suspending agents and thickening agents. Compositions may be presented in unit-dose or multi-dose containers, for example sealed ampoules and vials. Extemporaneous injection solutions and suspensions may be prepared from sterile powders, granules and tablets. Exemplary compositions include injectable solutions or suspensions which can contain, for example, suitable non-toxic, parenterally acceptable diluents or solvents, such as polyethylene glycol, ethanol, 1,3-butanediol, water, Ringer's solution, an isotonic sodium chloride solution, or other suitable dispersing or wetting and suspending agents, including synthetic mono- or diglycerides, and fatty acids, including oleic acid, or Cremophor®.

### BRIEF DESCRIPTION OF THE FIGURES

FIG. 1 is a schematic representation of the dosing schedule for the Phase 1b/2 investigation of combined APR-246 and azacitidine described in the Example.

FIG. 2 is a schematic overview of Phase 2 of the study of combined APR-246 and azacitidine described in the Example.

### EXAMPLE

### Clinical Study of APR-246 in Combination with Azacitidine

### Study Design

The study, identified as NCT03072043 on clinicaltrials.gov, is a multi-institution, open-label, phase Ib/II clinical trial conducted in 2 parts: a Phase 1 b part followed by a Simon's two-stage Phase 2 design. The study assesses the safety and efficacy of APR-246 in combination with azacitidine for the treatment of TP53 mutant myeloid neoplasms. All patients were pre-screened by NGS on peripheral blood (PB) or bone marrow (BM) samples to determine TP53 mutational status and therefore eligibility to participate in this study. A PB sample was obtained prior to treatment for NGS by a central laboratory, to evaluate baseline TP53 VAF and for serial analysis.

### APR-246 Administration

Treatment was administered on an outpatient basis. No investigational or commercial agents or therapies other than those described were administered with the intent to treat the patient's MDS or MDS/MPN. APR-246 was supplied by Aprea Therapeutics AB. The investigational medicinal product was a concentrate for solution, diluted with sterile 0.9% NaCl solution for infusion before administration. The solution for infusion was prepared with the prescribed dosage for each patient in accordance with the protocol and separate technical instruction.

Phase Ib Part

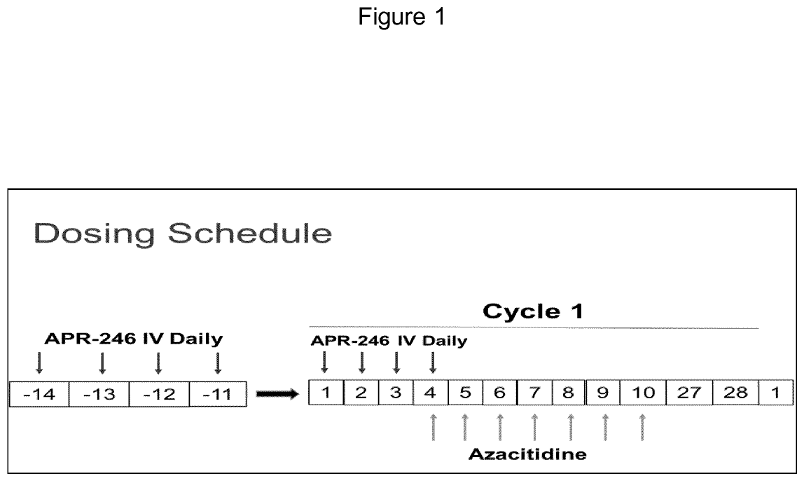

The initial Phase 1 b part evaluated APR-246 administered with azacitidine. Cohorts of at least 3 evaluable patients were enrolled using a modified 3+3 design. Patients received IV infusions of APR-246 as a lead-in phase on days −14 to −11, starting at Dose Level 1 (see below and in Table 1) prior to starting cycle #1 of combination therapy with azacitidine (see FIG. 1) In all cycles, APR-246 was administered as a 6-hour infusion daily for four consecutive days (study days −14 to −11 of the lead in phase and day 1 to 4 of each combination cycle).
Dose Level 1 (starting dose): APR-246 21.0 mg/kg LBM (for the first 45 min)+29 mg/kg LBM (for 5 h 15 min) on days 1 to 4.
Dose Level 2: APR-246 30.0 mg/kg LBM (for first 45 min)+45.0 mg/kg LBM (for 5 h 15 min) on days 1 to 4.
Dose Level 3: APR-246 37.0 mg/kg LBM (for first 45 min)+63.0 mg/kg LBM (for 5 h 15 min) on days 1 to 4.

The dosing schedule for the combined administration of APR-246 and azacitidine is shown in FIG. 1. Thus, the combination therapy consisted of APR-246 on days 1-4 and azacitidine on days 4-10 of a 28-day cycle. Azacitidine was administered subcutaneously (SC) or intravenously (SC being preferred) at 75 mg/m² for 7 days (either for 7 consecutive days (days 4-10) or on 2+5 days (i.e. days 4-5 and 8-12)). Azacitidine was administered on day 4 at the same time as the final infusion of APR-246. Azacitidine was given following the completion of APR-246 infusion. Cohorts of at least 3 evaluable patients were enrolled using a modified 3+3 design. Patients were followed for at least 6 weeks (lead-in phase+cycle 1) before decision to dose escalation in the next cohort following a full safety assessment. The MTD was defined as the dose level below which DLT is manifested in 33% of the patients or at dose level 1 if DLT is manifested in <33% of the patients. According to these criteria, the dose escalation Phase Ib was finalized after 12 patients, to reach a recommended Phase 2 dose of 100 mg/kg lean body weight.

TABLE 1
Dose levels for treatment part 1: APR-246 + azacitidine

| | APR-246 | Azacitidine |
|---|---|---|
| Dose level | (mg/kg LBM) | (mg/m²) |
| 1 (starting dose) | 50 | 75 |
| 2 | 75 | 75 |
| 3 | 100 | 75 |

### Rationale for Fixed Vs Body Weight Based Dosing

APR-246 was given in mg/kg body weight in the APR-246-01 study, as well as in the Phase Ib part of the APR 407 study (PiSARRO). After the population pharmacokinetic (“popPK”) evaluation based on partial data showed that LBM was a slightly stronger influence than weight on the pharmacokinetics of APR-246, the 67.5 mg/kg dose of APR-246 was converted and recalculated to 100 mg/kg LBM (37 mg/kg in the first infusion step and 63 mg/kg in the second) for the Phase 2 part of APR 407 (PiSARRO).

Subsequently, a more thorough evaluation of the effect of body size-based vs fixed dosing was conducted using an updated popPK model, and it was found that the effect of WT or LBM on the PK of APR-246 is weak. Simulations were performed and three different dosing regimens were compared:
1) LBM-based dosing: 100 mg/kg LBM split into 37 mg/kg LBM during the first 45 min, followed by 63 mg/kg LBM for 5 h 15 min.
2) Weight-based dosing: 67.5 mg/kg WT split into 25 mg/kg WT during the first 45 min, followed by 42.5 mg/kg WT for 5 h 15 min.
3) Fixed dose regimen: 4500 mg, split into 1500 mg for 45 min followed by 3000 mg for 5 h 15 min.

The fixed dose regimen of 4500 mg was chosen to match the mean AUC in females in the WT based dosing group. For each of the dosing regimens, 1000 individuals were resampled from the individuals in the analysis dataset. Summary statistics of the simulated Cₘₐₓ and AUC values for males and females were established. The plasma levels after a fixed dose of 4500 mg given to women (split as 1500 mg for the first 45 min and 3000 mg for the subsequent 5 h 15 min) predict an average AUC of 571 mg·h/l. The average simulated Cₘₐₓ value in women treated at this fixed dose is 57.5 mg/l. This is very close to the expected value at 67.5 mg/kg WT or 100 mg/kg LBM. Since the predicted PK variability is very similar, it is justified to apply fixed dosing of APR-246, as it will remove the risk for miscalculations of the dose and thereby contribute to the safe use of the product.

### Phase 2 Part: Simon's Two-Stage Minimax Design

Following completion of the dose finding phase described above, a dose expansion was carried out, whereby patients are treated with APR-246 administered at the MTD with azacitidine on a 28-day cycle with the same dosing schedule as in Phase Ib, with the exception of no lead in phase (i.e. patients will start at day 1 of combination therapy with APR-246 on days 1˜4 and azacitidine on days 4-10 (or days 4-5 and 8-12) of a 28-day cycle as illustrated in FIG. 1), and the exception that the recommended Phase 2 dose was modified from 100 mg/kg LBM to a fixed dose regimen of 4500 mg/patient (i.e. APR-246 administration in 2 consecutive steps as a loading dose (1500 mg for first 45 min) and a maintenance dose (3000 mg for 5 h 15 min)). At any dose reductions during the phase 2 part of the trial, administration of APR-246 was also given as a loading dose during 45 min and a maintenance dose during 5 h and 15 min (see below).

TABLE 2
Dose modification for APR-246

| Dose modification | APR-246 dose |
|---|---|
| Phase 2 Starting | 4.5 g/patient/day |
| Dose Level (DL) | 1.5 g/patient (for first 45 min ) + |
| | 3.0 g/patient (for 5 h 15 min) |
| First dose reduction | 4.0 g/patient/day |
| DL-1 | 1.33 g/patient (for first 45 min) + |
| | 2.67 g/patient (for 5 h 15 min) |
| Second dose | 3.5 g/patient/day |
| DL-2* | 1.16 g/patient (for first 45 min) + |
| | 2.34 g/patient (for 5 h 15 min) |

A Simon's two-stage minimax design was applied as follows:

Stage 1: Enrolment of a total of 24 evaluable patients at the MTD (including patients who were enrolled during the Phase 1 part of the study). More than 6 of 24 patients achieved CR, and enrolment was continued to Stage 2.

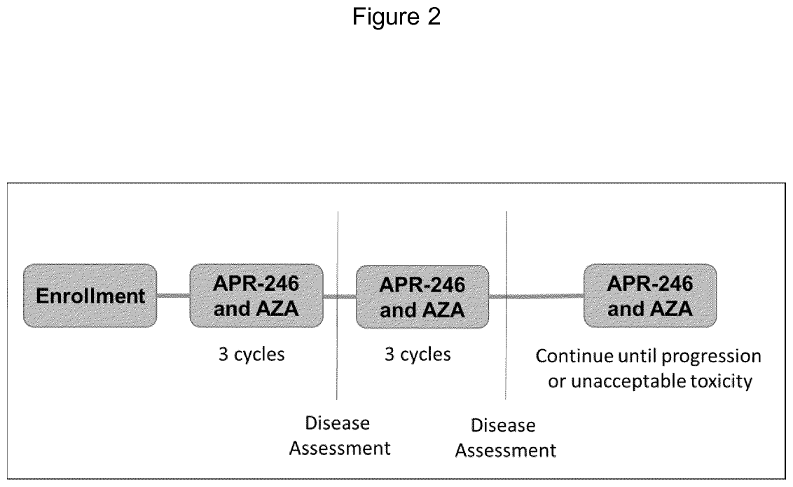

Stage 2: Enrolment of 21 more evaluable patients for a total of 45. More than 14 of 45 patients achieved CR, indicating that there is sufficient evidence to support further study of APR-246 in combination with azacitidine in Phase 3. Additional evaluable patients were treated at the level of the MTD in the Phase 2 portion of the trial. The total number of enrolled patients was 55, of whom 40 had a diagnosis of MDS. Disease assessment was performed after 3 cycles of therapy (FIG. 2).

### Duration of Therapy

Many subjects were treated for more than 6 cycles, due to stable disease or better, since treatment could continue until one of the following criteria applied: i) inter-current illness that prevents further administration of treatment; ii) unacceptable adverse event(s); iii) patient decides to withdraw from the study; iv) general or specific changes in the patient's condition render the patient unacceptable for further treatment in the judgment of the investigator; v) evidence of disease progression by the IWG 2006 criteria, not applicable at bone marrow evaluation at day −10 of lead-in phase.

Subjects who did not wish to continue treatment could complete their end of study visit upon completion of cycle 6.

### Duration of Follow-Up

Subjects were followed as per calendar on treatment for 6 cycles. After 6 cycles, patients who continued on treatment were followed monthly.

The following efficacy data was obtained from a data cut taken after the majority of patients had received, or had had an opportunity to receive, at least 6 months of treatment. The CR rate was 50% and ORR 73% in the MDS population (n=40). Among these 40 patients, 7 were not efficacy evaluable due to early discontinuation from treatment and prior to repeat bone marrow biopsy for reasons of: withdrawal/refusal of treatment (n=4), adverse event (n=2) and death (n=1). The response rates in the protocol-defined efficacy evaluable MDS population (n=33) were CR 61% and ORR 88%. Further, 40% of MDS patients (48% of efficacy evaluable) were able to discontinue therapy and proceed to allogeneic SCT.

1. Method of treatment of a neoplastic disease in a patient carrying a mutant TP53 gene, comprising
administration to said patient of APR-246 at a fixed dose within the interval 2.7-7.5 g, and
administration to said patient of azacitidine at a therapeutically effective dose.

2. Method according to claim 1, wherein said fixed dose of APR-246 is within the interval 3.5-6.0 g.

3. Method according to claim 2, wherein said fixed dose of APR-246 is within the interval 4.0-5.0 g.

4. Method according to claim 3, wherein said fixed dose of APR-246 is approximately 4.5 g.

5. Method according to claim 1, wherein said therapeutically effective dose of azacitidine is a body surface area based dose within the interval 70-80 mg/m².

6. Method according to claim 5, wherein said therapeutically effective dose of azacitidine is a body surface area based dose of 75 mg/m².

7. Method according claim 1, wherein said treatment is carried out pursuant to the following dosing scheme over a 28-day cycle:
a) daily administration of APR-246 for four days, for example four consecutive days;
and
b) daily administration of azacitidine for at least 7 days during the 28-day cycle.

8. Method according to claim 7, wherein said at least 7 days during the 28-day cycle in b) are 7 consecutive days.

9. Method according to claim 8, wherein said 7 consecutive days overlap with the days of APR-246 administration.

10. Method according to claim 9, wherein
a) said daily administration of APR-246 is carried out on days 1-4; and
b) said daily administration of azacitidine is carried out during 7 consecutive days selected from days 1-7, days 2-8, days 3-9 and days 4-10.

11. Method according to claim 7, wherein
a) said daily administration of APR-246 is carried out on days 1-4; and
b) said daily administration of azacitidine is carried out during 7 days selected from days 1-5 and 8-9; days 2-5 and 8-10; days 3-5 and 8-11; and days 4-5 and 8-12.

12. Method according to claim 1, wherein said administration of APR-246 is parenteral.

13. Method according to claim 12, wherein said administration of APR-246 is performed as an intravenous infusion.

14. Method according to claim 1, wherein said administration of azacitidine is parenteral.

15. Method according to claim 14, wherein said administration of azacitidine is performed as a subcutaneous injection.

16. Method according to claim 14, wherein said administration of azacitidine is performed as an intravenous infusion.

17. Method according to claim 1, wherein said neoplastic disease is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

18. Method according to claim 17, wherein said neoplastic disease is selected from malignant neoplasms of lymphoid, hematopoietic and related tissue including multiple myeloma, lymphoid leukemia or myeloid leukemia; and neoplasms of uncertain or unknown behavior including myelodysplastic syndrome.

19. Method according to claim 18, wherein said neoplastic disease is myelodysplastic syndrome.

20. Pharmaceutical dosage form, comprising APR-246 at a fixed dose within the interval 2.7-7.5 g and at least one pharmaceutically acceptable excipient.

21. Pharmaceutical dosage form according to claim 20, in which said fixed dose is within the interval 3.5-6.0 g.

22. Pharmaceutical dosage form according to claim 21, wherein said fixed dose of APR-246 is within the interval 4.0-5.0 g.

23. Pharmaceutical dosage form according to claim 22, wherein said fixed dose of APR-246 is approximately 4.5 g.
